(19) United States
(12) Patent Application Publication
(10) Pub. No.: US 2010/0297779 A1
(43) Pub. Date: Nov. 25, 2010

(54) Biochip Self-Calibration Process

(75) Inventor: Michael Thomas Georges Canva, Gif S/Yvette (FR)

Correspondence Address: LERNER, DAVID, LITTENBERG,;KRUMHOLZ & MENTLIK, 600 SOUTH AVENUE WEST, WESTFIELD, NJ, 07090, US

(73) Assignees: CENTRE NATIONAL DE LA RECHERCHE SCIENTIFIQUE (CNRS), PARIS (FR); UNIVERSITE PARIS-SUD, ORSAY (FR)

(21) Appl. No.: 12/086,529

(22) PCT Filed: Dec. 13, 2006
(86) PCT No.: PCT/EP2006/069683

§ 371 Date: May 14, 2009

(30) Foreign Application Priority Data
Dec. 13, 2005 (FR) 0512594

(51) Int. Cl. G01N 33/543; G01N 30/96

(52) U.S. Cl. 436/518; 422/69

(57) ABSTRACT

The invention relates to a calibration process for determining the presence and/or the amount of a target compound in a test sample, using a solid support, attached to the surface of which are a compound capable of binding specifically with said target compound and a calibration probe compound, the molar ratio of said compound Csc to said compound Cs being known, in particular for nucleic acid or protein biochips. The invention also comprises the use of such a support for the self-calibration of a measurement, in particular by surface plasmon resonance or by fluorescence, and also a device or a kit comprising such a support and a standard calibration reagent.

### BACKGROUND OF THE INVENTION

This invention relates to an internal calibration process for determining the presence and/or the amount of a target compound in a test sample, said process using a solid support attached to the surface of which is a calibration probe compound, namely for SPR imaging or fluorescence imaging. This invention also includes a kit and device for implementing such a process.

Many techniques and devices for the analysis of biological samples have been developed over recent years, in particular for parallel analysis of large quantities of nucleic acids or proteins, notably following the rapid development of genome and proteomic technology. Thus DNA or protein chip technology, or biochips, is currently undergoing and exceptional expansion which has resulted in a great deal of interest from the scientific community. The understanding of the level of expression of a gene in these different situations constitutes an advance towards functional understanding but also towards the targeting of new molecules and the identification of new medicinal products and diagnostic tools.

These techniques and devices include supports allowing high-rate analysis of nucleic acids or peptides such as biochips or DNA or protein chips (also called micro- or macroarrays) which have the subject of many studies.

In particular, these biochips can be produced from a solid support which is functionalised and to which given nucleic acids or peptides are attached (nucleic probes or peptide probes) and to which nucleic or peptide probes then bind specifically by pairing (or specific hybridization) or by recognition of target nucleic acid or protein binding sites which are to be detected, identified and/or quantified in a biological sample.

Among the documents describing techniques relating to DNA biochips, we can cite in particular:
the review article by Wang J. (Nucleic Acids Research, 28, 16, 3011-3016, 2000), which presents an up-to-date summary of the main known techniques for DNA chips and the document by Schubhart et al. (Nucleic Acids Research, 28, 10, e47, 2000) which lists the problems facing chip designers;
U.S. Pat. No. 6,030,782 which describes grafting with a mercaptosilanised surface of nucleic acids modified by a sulhydryl or disulphide group, and the article by Bamdad (Biophysical Journal, 75, 1997-2003, 1998) which describes how to obtain surfaces with DNA by incorporation of composite molecules, DNA-thiols, into self-assembled monolayers or SAMs;
international patent application No WO 00/43539 which suggests immobilising molecules, such as oligonucleotides, by means of polyfunctional polymers which make it possible to increase the density of grafting. These polymers can be obtained from monomers such as hydroxyethyl methacrylate, acrylamide or vinyl pyrrolidone;
international patent application No WO 00/36145 describes a method for the manufacture of DNA chips comprising polymerisation on a metal layer type substrate of a pyrrole or functionalised pyrrole copolymer, fixation of a reticulating agent on the functionalised pyrrole followed by fixation of a biological probe (such as an oligonucleotide). The reticulating agent can be bifunctional and can, for example, have an N-hydroxysuccinimide ester function and a maleimide function;
international patent application No WO 98/20020 which also describes high density immobilisation of nucleic acids on solid supports, this time by contacting a nucleic acid containing a thiol group with a support presenting a group which reacts with this thiol, possibly through the intermediate of a reticulating agent.

Amongst the techniques or devices for analysis of biological samples which have also been developed over recent years for parallel analysis of nucleic acids or proteins, we can also cite surface plasmon resonance (SPR), a technique which over the past few years has become a well-accepted analytical tool for monitoring interfacial processes as well as the characterisation of thin films. It can be noted that this technique offers the advantage of not requiring labelling of the specific complex formed between the probe attached to the support and the target compound which is to be detected, identified and/or quantified. In this SPR technique, the sensitivity of the method results from stimulation of electromagnetic fields, surface plasmon waves, created on a metal-dielectric interface. This localisation of the luminous field in close proximity to the interface limits light-matter interactions at low volume, the slightest modification in which has important consequences on the properties of surface plasmon. Such suprafacial plasmons are excited on gold surfaces when polarised light TM illuminates the gold/dielectric interface by means of a prism under total reflection, coupling incident light under a certain angle in surface plasmon modes or via a diffraction network. The plasmon formation is correlated to a marked reduction in reflected light which can be measured by a photodiode. The changes to the surface state, notably those linked to normal functioning of a biochip, lead to modifications in the state of the surface plasmon waves and therefore in their coupling efficacy in a given optical configuration. As a result, targets which hybridise can be quantified, namely by measuring the change in angle or resonance wavelength, or even simply by a change in reflectivity. These changes are extremely sensitive to any alteration in the refraction index (n) of the adjacent medium and any change in optical thickness. For example, gold and silver are ideal candidates for metal films in an SPR chip in the region of visible light. The SPR technique has been widely used for label-free detection, the study of DNA hybridization reactions and detection of molecular and biomolecular events in real time. This results from the fact that the principle of detection is based on changes in optical contrast brought about by a molecule bound to the interface in comparison to the neighbouring environment. The chemistry used for immobilisation of biological compounds on the surface of gold on an SPR chip is principally based on the use of thiol compounds (see for example Peterlinz K. A. et al., Am. Chem. Soc., 1997, 119, 3401-3402; Smith E. A. et al., Langmuir, 2001, 17, 2502-2507; Smith E. A. et al., Am. Chem. Soc., 2003, 125, 6140-6148; Damos F. S. et al., Langmuir, 2005, 21, 602-609) or conducting polymers (see for example Guedon P. et al., Anal. Chem., 2000, 72, 6003-6009; Jung L. S. et al., J. Phys. Chem. B., 2000, 104, 11168-11178; Szunerits S. et al., Langmuir, 2004, 20, 9236-9241). The Biocore company manufactures bioanalytical systems based on the SPR phenomenon (see: http//www.biacore.com.). In this system, a functionalised dextran layer is coupled to a gold surface to bind various chemical and biological species to the surface.

In the context of post-genomic technology, there is an urgent need to have available routine biochip systems outside the environment of research laboratories. Before achieving this, it is necessary to obtain better characterisation and understanding of the materials and compounds currently used in order to improve the quality and performance of biochip systems.

At present, technical platforms using these biochips are mainly found in research laboratories. Interpretation of the measurements obtained still requires specialist involvement given the high level of “noise” and/or “fluctuation” associated with these measurements which makes quantitative interpretation difficult. In the particular context of genetic diagnosis, there is also a lack of reliability in large-scale genotyping (automated) which has until now prevented low-cost routine use. This gap has therefore not been filled at present.

The accuracy of measurements is currently restricted by partial mastery of the functionality of the surfaces used. In the case of use of biochip plots (a plot or spot corresponds to a distinct surface of the chip where the same entities are deposited), this is seen spatially by the absence of homogeneity between the different plots and a great deal of heterogeneity between them. This means that the differences that are being investigated are smaller than the intrinsic dispersions of biochip surfaces.

This is why an allowing self-calibration of each of the pixels of the obtained images to be made available in order to significantly improve measurement of the reactivity of biochemical interactions which take place at these sites.

Such a method may involve a very slight increase in the cost of the preparation of biochips and in the protocol for their use while allowing a significant increase in accuracy, and consequently in of the density of information, at equal performances.

This is precisely the subject of the present invention.

### BRIEF DESCRIPTION OF THE DRAWINGS

FIG. 1: FIG. 1 is a diagram of an example of the structure of a multifunctional probe according to the invention represented in functional units where local measurement of the effective accessible concentration of the calibration group (Csc) is directly proportional to the effective accessible concentration of the specific functional group (Cs).

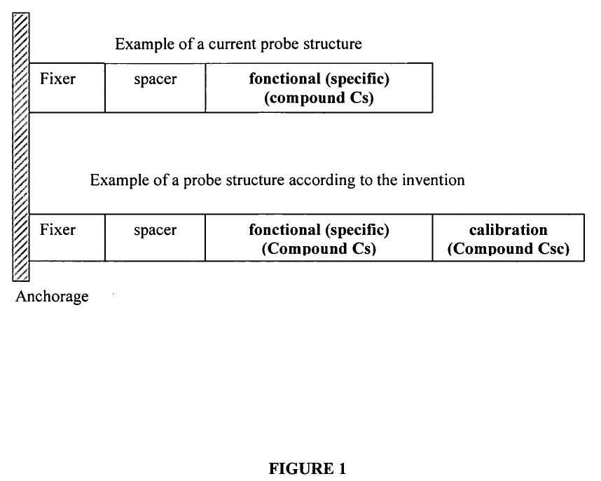

FIGS. 2A and 2B: Example of 100 supposedly identical measurements but whose values are dispersed, notably as a result of spatial variations in accessible probe concentrations. FIG. 2B represents the actual values obtained for the histograms shown in FIG. 2A.

FIGS. 3A and 3B: Histogram associated with FIGS. 2A and 2B above. The raw measurements corrected by the calibration measurements are significantly less dispersed. FIG. 3B represents the actual values obtained for the histograms shown in FIG. 3A.

### DETAILED DESCRIPTION OF THE INVENTION

The inventors have found that it is possible, particularly in the case of DNA biochips, to introduce into the same plot, and if need be into several plots, a calibration probe compound (Csc) specific to a calibration target compound (Ccc) in addition to the probe compound (Cs) specific to the target compound (Cc). A single passage of the solution containing compound Ccc makes it possible to quantify the effective density of accessible Cs probes for each of the pixels, a value which is then used as an individual normalisation parameter. With such a process, the inventors have found that it is possible to significantly reduce the dispersion of measurements made for each pixel in a same plot or between several complementary plots, thus leading to an improvement and greater accuracy of measurement involving these biochips.

With such a process, the inventors have found that it is possible to carry out self-calibration of the measurements obtained for each of the pixels of the biochip, thus making it possible to overcome the majority of fluctuations caused, in raw measurements, by the lack of homogeneity of the materials and compounds used.

The biochip self-calibration process according to the invention is based on:
sequential reading by a dynamic reading method of the probe:target complex formation reactions on a previously functionalised and structured surface; and
adequate functionalisation of this surface by multi-functionalised probes comprising at least one calibration probe and one specific probe.

Thus the present invention relates to a self-calibration process for measurements carried out on a solid support (biochip) for determining the presence and/or quantity of a target compound Cc in a test sample, said process using a solid support to whose upper surface is attached a probe compound Cs capable of binding specifically to said compound Cc likely to be contained in the test sample, said process comprising:
a step a) consisting of contacting the test sample likely to contain said compound Cc with said support under conditions allowing the formation of a specific complex Cc/Cs; and
a step b) for measuring the formation of a specific complex Cc/Cs, possibly formed in step a), preferably by means of a dynamic label-free measurement method wherein:
said solid support also includes attached to this upper surface a calibration probe compound Csc capable of binding specifically to a calibration target compound Ccc, the molar ratio of said Csc compound to said compound Cs being known, and wherein the process is comprised of the following steps prior to or after steps a) and b):
c) contacting a sample of said compound Ccc with said support under conditions allowing the formation of a specific complex Csc/Ccc; and d) measurement of the formation of a complex Csc/Ccc of step c) by the measurement method used in step b),
determination of the presence and/or quantity of said target compound Cc in the test sample being based on the measurements obtained in step b) and step d).

The conditions allowing specific formation of the complex Cc/Cs or Ccc/Csc, notably a specific nucleic acid/nucleic acid complex or polypeptide/polypeptide complex is well known in the art and will not be described here. For example, in the case of nucleic acid complexes, the conditions allowing specific hybridization of said target nucleic acids with said nucleic acid probes will be described here. These are preferably high stringency conditions, in particular as described hereafter.

High stringency hybridization conditions mean that the temperature and ionic force conditions are chosen such that they support hybridization between two complementary DNA or RNA/DNA fragments. As an example, high stringency conditions in the hybridization step aimed at defining the hybridization conditions described above are advantageously as follows.

DNA-DNA or DNA-RNA hybridization is carried out in two steps: (1) prehybridization at 42° C. for 3 hours in phosphate buffer (20 mM, pH 7.5) containing 5×SSC (1×SSC corresponds to a solution of 0.15 MNaCl+0.015 M sodium citrate), 50% formamide, 7% sodium dodecyl sulfate (SDS), 10×Denhardt's, 5% dextran sulphate and 1% salmon sperm DNA; (2) hybridization for 20 hours at a temperature dependent on the size of the probe (i.e. 42° C., for a probe with a size >100 nucleotides) followed by 2×20 minutes washing at 20° C. in 2×SSC+2% SDS, 1 washing for 20 minutes at 20° C. in 0.1×SSC+0.1% SDS. The latter washing is carried out in 0.1×SSC+0.1% SDS for 30 minutes at 60° C. for a probe with a size >100 nucleotides The high stringency hybridization conditions described above for a nucleotide of a given size can be adapted by the man skilled in the art for larger or smaller oligonucleotides according to the description of Sambrook et al. (1989, Molecular cloning: a laboratory manual. 2nd Ed. Cold Spring Harbor).

For Cc/Cs or Ccc/Csc complexes of the polypeptide/polypeptide type, this is most commonly a specific complex obtained by affinity between two polypeptides (ligand/receptor, antigen/antibody, etc). The conditions allowing the formation of such affinity complexes are also well known, notably in terms of incubation temperature (between 20 and 37° C.), molarity, pH and/or salinity of the buffers used (PBS type buffer at pH7.4).

According to a preferred embodiment, the process according to the invention is characterised in that:
when steps c) and d) are carried out prior to steps a) and b), the solid support is washed under appropriate conditions after step d) in order to eliminate compounds Ccc of complex Csc/Ccc from the solid support; or
when steps c) and d) are carried out after steps a) and b), the solid support is washed under appropriate conditions after step b) in order to eliminate compound Cc from complex Cs/Cc from the solid support.

Depending on the nature of the complex obtained (nucleic acid/nucleic acid, polypeptide/polypeptide or nucleic acid/polypeptide), the man skilled in the art has knowledge of the conditions and solutions to be applied in order to denature such a complex.

For example, and without this being limiting in any way, a rinsing solution at around 80° C. can be used to denature a nucleic acid duplex formed for complex Csc/Ccc or Cs/Cc. Commonly used denaturation conditions can also be used to obtain a probe compound of a single strand type nucleic acid Cs from a double strand type probe compound attached to the support (see for example Peterson et al., Nucleic Acids Research, 29 (24), 5163-5168, 2001, Materials and Method).

For polypeptide/polypeptide type complexes, elimination of the Cc or Ccc compound of the complex formed can be carried out with rinsing solutions whose pH and/or ionic force will be adjusted so as to bring about denaturation of the complex. Such methods are commonly used in affinity chromatographic techniques to elute the polypeptides of interest complexed by affinity to another polypeptide attached to a chromatography support.

While washing the solid support after steps d) or b) in order to remove the Ccc or compound Cc of the complex Csc/Ccc or Cs/Cc from the solid support can be advantageously used for any type of measurement system, it is particularly advantageous to carry out washing when a measurement system requiring labelling of Cc or Ccc target compounds using the same label is necessary.

According to a preferred embodiment, the process according to the invention is characterised in that steps c) and d) are carried out prior to step a).

It can be advantageous, particularly when probe compounds Cs and Csc are nucleic acids, to attach spacer arms between the support and the probe compound to which are attached probe compounds in order to give the grafted probe mobility. For short sequence oligonucleotides in particular, the nucleic acids grafted onto the support (by adsorption or covalent coupling) are not always fully accessible to the target sequence during hybridization. The spacer compound is generally attached to the extremity of the probe compound initially intended for attachment to the support. The nature of this compound can vary. It can be a nucleic acid sequence with no homology with the target sequences Cc or Ccc or their complementary sequences or it can be a polyethylene glycol type compound (see patent WO 03/068712).

Thus according to a preferred embodiment of the invention, the spacer compound is attached to said support and compound Cs and compound Cc are attached to the support independently of each other by means of said spacer compound.

According to a preferred embodiment, the process according to the invention is characterised in that Csc is attached to compound Cs by a covalent bond.

According to a preferred embodiment, the process according to the invention is characterised in that compound Csc is attached to the free, non-attached extremity of the support of compound Csc.

According to a preferred embodiment, the process according to the invention is characterised in that compound Csc is attached to the surface of the solid support independently of compound Cs, the molar ratio of said attached compound Csc to said attached compound Cs being known.

The term independently refers here to the fact that compound Csc is not attached directly to compound Cs.

According to a preferred embodiment, the process according to the invention is characterised in that compound Csc and compound Cs are attached to the surface of the solid support by fixation to the same molecule, with the latter being attached to the support.

According to a preferred embodiment, the process according to the invention is characterised in that the same molecule to which compound Csc and compound Cs are attached is a polymer such as, but without this being limiting in any way, a pyrrole or polyethyleneimine polymer coupled to several avidines.

For example, for systems using measurements by SPR imaging, we can refer to the fixation method by electropolymerisation of pyrrole residues carrying a probe compound (Cs or Csc) on a glass slide coated with a metal surface (Maillart et al., Oncogene, p. 1-8, 2004, et Guedon et al., 2000) or the method using polyethyleneimine coupled to avidine with a biotinylated probe compound (Bassil et al., Sensors and Actuators, B 94, 313-323, 2003).

According to a preferred embodiment, the process according to the invention is characterised in that:
the same Cs and Csc compounds are attached to a same defined surface (called plot or spot) on the support;
the sample of compound Ccc and the test sample likely to contain compound Cc of step c) are contacted with said plot on the support;
measurements in step b) and step d) are carried out for a set of points or pixels on said plot; and
determination of the presence and/or quantity of said target compound Cc in the test sample is carried out taking into consideration the set of measurements obtained in step b) and step d) for each of the measured points or pixels on said plot.

According to a preferred embodiment, the process according to the invention is characterised in that for a same plot, the conditions allowing specific formation of the complex Cc/Cs and Ccc/Csc are identical.

According to a preferred embodiment, the process according to the invention is characterised in that said support comprises n plots, n being between 2 and 10⁸, preferably between 2 and 10⁷, between 2 and 10⁶, between 2 and 10⁵, between 2 and 10⁴, between 2 and 10³ or between 2 and 10³ plots.

According to a preferred embodiment, the process according to the invention is characterised in that the Csc and Ccc compounds used are identical for n plots.

According to a preferred embodiment, the process according to the invention is characterised in that Csc and Ccc compounds used are different for at least 2 plots.

According to a preferred embodiment, the process according to the invention is characterised in that compound Cs used and compound Cc investigated are different between at least 2 plots.

According to a preferred embodiment, the process according to the invention is characterised in that the conditions allowing the specific formation of the complex Cc/Cs or Ccc/Csc are identical for 2 plots between which the compound Cs used and the compound Cc investigated are different, preferably between all plots on the solid support.

According to a preferred embodiment, the process according to the invention is characterised in that said compound Cs is chosen from among the group of compounds made up of nucleic acids, peptides-nucleic acids (PNA), polypeptides, oligosaccharides, lipids, preferably nucleic acids, PNA and polypeptides.

According to a preferred embodiment, the process according to the invention is characterised in that said compound Cs and said compound Cc are chosen from the following pairs (Cs, Cc):
(nucleic acid, nucleic acid)
(nucleic acid, polypeptide)
(polypeptide, nucleic acid)
(polypeptide, polypeptide)

The preferred (Cs, Cc) pair being (nucleic acid, nucleic acid).

According to a preferred embodiment, the process according to the invention is characterised in that:
when said compound Cs and said compound Cc are nucleic acids, compounds Csc and Ccc are nucleic acids.
when said compound Cs and said compound Cc are polypeptides, compounds Csc and Ccc are polypeptides and
when said compound Cs is a nucleic acid and said compound Cc is a polypeptide, compound Csc is a nucleic acid.

According to a preferred embodiment, the process according to the invention is characterised in that said compound Cs and said compound Cc are nucleic acids, notably chosen from the nucleic acid group made up of double strand DNA, single strand DNA or even a mixed single and double strand type of nucleic acid, with the product of transcription of said DNAs, such as RNAs, as well as any nucleic acid including or not non-natural nucleotides.

According to a preferred embodiment, the process according to the invention is characterised in that compound Csc attached to a plot on the solid support is a nucleic acid with no significant homology to attached compound Cs and investigated compound Cc or their complementary sequence on this plot.

The term no significant homology means here a degree of identity between the sequences such that this does not allow hybridization between these sequences under the specific hybridization conditions used in step a) of contacting the test sample likely to contain said compound Cc with said support under conditions allowing the specific formation of complex Cc/Cs, and in step c) contacting a sample of said compound Ccc with said support under conditions allowing specific formation of complex Csc/Ccc.

According to a preferred embodiment, the process according to the invention is characterised in that compound Csc and, if necessary, compound Ccc is a nucleic acid with a length of 6 to 30 nucleotides, preferably 6 to 24 nucleotides, 6 to 20 nucleotides, 6 to 15 nucleotides or 6 to 12 nucleotides.

According to a preferred embodiment, the process according to the invention is characterised in that said support is a solid support preferably chosen from among the following supports: glass, silicon, silicone, Kevlar™, polymer (such as plastic, polyacrylamide, polypyrrole, polyoses), metals (gold, platinum). When the measurement system used is SPR imaging, these supports, particularly glass supports, are coated on one surface with a thin layer of metal (for example gold or silver).

These supports as well as the fixation methods for probe compounds Cs and Csc are well known to the man skilled in the art.

Among these supports, we can cite those requiring labelling of the target for their detection, for example:
Macroarray type supports
Support: nylon membrane; spot size: 0.5-1 mm, density: several hundred spots/cm²; probes: for example PCR or synthetic products; targets: for example cDNA with radioactive labelling with ³²P;
spotted microarray type supports
Support: glass slide with a chemical coating, spot size: ˜100 μm; density: 1000-10000 spots/cm²; probes: for example PCR or synthetic oligonucleotides (20-70 mers); targets: for example cDNA or PCR products with fluorescent labelling (with Cy3 and Cy5);
Affymetrix gene chip type supports
Support: glass slide with chemical coating; spot size: ˜20 μm; density: up to 250000 spots/cm²; probes: for example 20-25 mers oligonucleotides synthesized in situ; targets: cRNA or PCR product with fluorescent labelling with biotine-streptavidine.

The methods for manufacturing spotted chips are now well established (DeRisi et al., Science, 278(5338):p. 680-686, 1997). When the target compounds are DNA type nucleic acids, DNA solutions are prepared either by PCR amplification or by oligonucleotide synthesis. Microdroplets of these solutions are then deposited by a robot, according to a defined placement matrix, on a glass slide treated with a chemical layer which allows the DNA probe to be attached to each spot (or plot) on the matrix.

The methods for manufacturing oligonucleotide chips synthesised in situ by photolithography are also well known. These include GeneChips™ technology developed by Affymetrix (Lipshutz R. J. et al., Nat. Genet., 1999, 21(1 Suppl):p. 20-24) or inkjet impression developed by Agilent Technologies/Rosetta Inpharmaceutics (Hughes T. R. et al., Nat. Biotechnol, 2001, 19(4):p. 342-347).

In the biochip self-calibration process of the invention, reading in step b) and d) can be carried out by any dynamic reading method, preferably by any label-free dynamic reading method.

The label-free dynamic reading methods which can be used in the biochip self-calibration process of the invention, includes the following methods which are in no way limiting:
Surface Plasmon Resonance (SPR);
use of guided waves, not at the metal:dielectric interface but around the high optical index guiding zone;
use of surface acoustic waves;
use of integrated electronic compounds;
use of impedance;
use of magnetoresistance; or even
use of deflector micro-mirrors.

These methods are well known to the man skilled in the art. For example, the following documents can be referred to:
Dharuman et al., <<Label-free impedance detection of oligonucleotide hybridization on interdigitated ultramicroelectrodes using electrochemical redox probes>>, Biosens. and Bioelect. 21, 645-654, 2005;
Cheng et al., “An array-based CMOS biochip for electrical detection of DNA with multilayer self-assembly gold nanoparticles”, Sensors and Actuators, B109, 249-255, 2005;
Nikitin et al., “Picoscope, a new label-free biosensor”, Sensors and Actuators B 111-112, 500-504, 2005; or even
in the general review document <<Emerging tools for real-time label free detection of interactions on functional protein microarrays>> by Ramachadran et al. (FEBS Journal (Federation of European Biochemical Society) volume 272, pages 5412 to 5425, 2005) as well as the documents cited as references in this document for implementation of these methods with supports adapted to the methods.

Compared to the SPR method, the other label-free dynamic reading methods cited above only require minor changes in the functionalisation of surfaces prior to depositing multifunctionalised probes, this in particular with the support used for the chosen reading method. The processes or protocols to be implemented for their use are identical to those of the SPR method (where reading requires the use of a metal film).

According to a preferred embodiment, the process according to the invention is characterised in that said support is a transparent solid support, preferably a glass slide when measurement of the formation of complexes in steps b) and d) is carried out by reading a signal on the lower surface of the solid support (opposite to the surface of the support where the probe compounds are attached), particularly when the process according to the invention is characterised in that measurement of the formation of complexes in steps b) and d) is carried out by surface plasmon resonance imaging (SPR).

Another embodiment of the process according to the invention includes processes in which compound Cc and compound Ccc are labelled.

When the process of the invention uses a measurement system requiring preliminary labelling of the target compounds (I.e. measurement of a fluorescent or radioactive signal), the target compounds Cc and Ccc which are to be detected and/or quantified undergo preliminary labelling with a label capable of generating a detectable signal whether directly or indirectly, preferably detectable by fluorescence.

Preferably, compounds Cc and Ccc undergo preliminary labelling with a different label, notably when measurement of complex Cs/Cc is carried out in the presence of the complex Csc/Ccc or vice versa.

Thus according to a preferred embodiment, the process according to the invention is characterised in that said label is a fluorescent label.

Preferably, when said labels are fluorescent labels, they are chosen from among cyanine derivatives, preferably cyanine sulphonate derivatives, namely the compounds Cy5 or Cy3, fluorochromes from the Alexa Fluor™ range (Molecular Probes Inc., U.S.A), or even with rhodamine or its derivatives.

According to a preferred embodiment, the process according to the invention is characterised in that said labels used to label compounds Cc and Ccc are identical and characterised in that:
when steps c) and d) are carried out before steps a) and b), the solid support is washed under suitable conditions after step d) in order to eliminate compound Ccc of complex Csc/Ccc from the solid support; or
when steps c) and d) are carried out after steps a) and b), the solid support is washed in suitable conditions after step b) in order to eliminate compound Cc of complex Cs/Cc from the solid support.

According to a preferred embodiment, the process according to the invention is characterised in that said labels used to label compounds Cc and Ccc are different.

For example, when the labels are of the fluorescent type and compounds Cc and Ccc are nucleic amino acids, compounds Cc and Ccc can be respectively labelled with Cy3 and Cy5 or vice versa.

The labelling of nucleic compounds with fluorescent labels is well known to the man skilled in the art and will not be described here. For example, the following can be cited without in any way being limiting when the desired target is genomic DNA: the labelling technique used is DNA polymerase (Klenow type enzyme) for synthesis of a complementary strand in the presence of labelled nucleotides (for example Cy3/Cy5 dCTP), or when the desired target is an RNA, the indirect labelling technique is used with reverse transcriptase (RT) in the presence of amino allyl dUTP.

Another aspect of the invention covers the use of a solid support comprising, attached to its upper surface, a first probe compound Cs capable of binding specifically to a target compound Cc likely to be contained in the sample, and a second probe compound Csc capable of binding specifically to another target compound Ccc, the molar ratio of said compound Csc to said compound Cs being known, said solid supports being used to determine and/or quantify the presence of target compound Cc by means of a system allowing measurement of the formation of a specific complex Cs/Cc by a dynamic reading method, preferably label-free, for calibration or self-calibration of said measurement carried out at the upper or lower surface of said support after contacting the sample likely to contain the target compound Cc with the upper surface of the support.

Preferably, use of a solid support according to the invention is characterised in that said compound Csc and said compound Cs are independently attached to the upper surface of said support by the same process, even more preferably, said compound Csc is attached to the upper surface of said support by covalent coupling to the extremities of compound Cs.

According to preferred embodiments, use of the support according to the invention is characterised in that said support and said compounds Cs, Csc, Ccc and Cc moreover have the characteristics defined independently in the above-described process of the invention.

Preferably, use of a solid support according to the invention is characterised in that said support is coated with a metal layer and in that the label-free dynamic reading method used is SPR imaging.

According to yet another aspect, the present invention covers a kit or the necessary equipment for calibration or self-calibration of a measurement, or kit or necessary equipment for determination and/or quantification of the presence of a target compound Cc in a sample by means of a system for measurement of the formation of a specific complex between said compound Cc and a probe compound Cs attached to the upper surface of a solid support, said compound Cs being capable of binding specifically to said target compound Cc, characterised in that measurement of the formation of complexes is carried out by means of a dynamic reading method, preferably label-free, and in that the kit or necessary equipment comprises:
a)—a solid support to which is attached at the upper surface a probe compound Cs and a calibration probe compound Csc different from said probe compound Cs, the molar ratio of said compound Csc to said compound Cs being known, or if necessary
   a solid support, a reagent Rs comprising compound Cs and reagent Rc comprising compound Csc, the two compounds Cs and Csc being used to for fixation to the upper surface of said support in a known molar ratio, and
b) a reagent Rcc comprising a calibration target compound Ccc, preferably of known concentration, if need be in a dry form for reconstitution or in solution, compound Ccc being capable of binding specifically to said compound Csc.

Preferably, the kit or necessary equipment according to the invention is characterised in that compound Csc is coupled by covalent bonds to one of the extremities of compound Cs, preferably the free extremity not attached to the support of compound Cs.

Preferably, compounds Cs and Csc are nucleic acids.

According to preferred embodiments, the kit or necessary equipment according to the invention is characterised in that said support and said compounds Cs, Csc, Ccc and Cc moreover have the characteristics defined independently in the above-described process of the invention.

Preferably, the kit or necessary equipment according to the invention is characterised in that said support is coated with a metal layer and in that the label-free dynamic reading method used is SPR imaging.

According to a final aspect, this invention comprises a device for measurement of the specific formation of a compound Cc on a solid support to which is attached at the upper surface a probe compound Cs capable of binding specifically to said target compound Cc characterised in that:
said solid support also comprising attached to its upper surface a calibration probe compound Csc different from compound Cs, capable of binding specifically to a calibration target compound Ccc, different from compound Cc, the molar ratio of said compound Csc to said compound Cs being known, said compound Csc and said compound Cs being attached independently to the upper surface of said support by a same process or said compound Csc being attached to the upper surface of said support by covalent coupling to one of the extremities of compound Cs;
a reagent comprising a calibration target compound Ccc, if need be in a dry form for reconstitution or in solution, capable of binding specifically to said compound Csc; and
a reading system using a dynamic reading method of the upper or lower surface of said support allowing measurement of the formation of specific complexes Cs/Cc and Csc/Ccc obtained at the upper surface of the support.

According to preferred embodiments, the device according to the invention is characterised in that said support, compounds Cs, Csc and Ccc and the reading system moreover have the characteristics defined independently in the above-described process of the invention.

Preferably, the device according to the invention is characterised in that said support is coated with a metal layer and in that the label-free dynamic reading method is SPR imaging.

Use of the kit or device according to the invention in the health sector for genotyping of identified mutations in order to obtain a comparative gene expression profile but equally, for example, health monitoring in terms of traceability and quality control of GMOs (genetically modified organisms) also falls within the scope of this invention.

Other characteristics and advantages of the invention will become apparent through the examples and figures whose captions are given below.

### EXAMPLES

### Example 1

Assembly of a solid support for SPR imaging with a nucleic type probe specific to target compound Cs attached to its surface, coupled to a calibration probe Csc.

Materials and methods
Example of adequate functionalisation in the case of dynamic DNA/DNA biochips.

We produced chips from a glass substrate, of the microscope slide type, onto which was deposited a layer of chromium of about 2 nm and a gold layer of about 50 nm. A molecular self-assembly system of the MUA (11-mercaptoundecanoic acid)/PEI (polyethylineimine)/extravidine type was added to this deposit as described in the document by Bassil et al., 2003, or of the 11-mercaptoundecanol/Dextran/avidine type (Biocore). As the final layer is rich in avidine groups, it is particularly well suited to deposits of new groups functionalised with biotine (the avidine/biotine complex is particularly stable). Biochips were functionalized by spotting biotinylated probe sequences (the probe sequences were diluted to a concentration of 7 μM in a PBS 1× and 1.5 M betaine solution). All the products used are commercially available.

The Csc calibration sequence in this example is introduced by means of covalent coupling at the 3′extremity of the probe Cs sequence for the set of probe plots.

For example, in order to characterise the genotype of the “MV470” mutation of exon n10 in the CFTR gene linked to mucoviscidosis, structures with the following sequences were used.

Mutations: fixing (5′ Biotin)+spacer ((T16)+target sequence+calibration (gac cgg tat gcg), that is for the probe compound Cs coupled to the calibration compound Ccc, respectively for the non-mutated gene (Wild type) M470V-WT, mutated gene (Mutated type) M470V-MT and the negative control:

M470V-WT
5′ Biotin (T)16 TTC TAA TGA TGA TTA gac cgg tat
gcg 3′
M470V-MT
5′ Biotin (T)16 TTC TAA TGG TGA TTA gac cgg tat
gcg 3′
Negative control
5′ Biotin (T)18 CAC TTC GTG CCT T gac cgg tat
gcg 3′

The calibration sequences can be taken as soon as their lengths are such that the complementary duplexes formed are sufficiently stable and preferably do not present cross-reactions with other sequences used in the biochip. In order to simplify the process of choosing such a calibration probe sequence, they can be selected from those introduced as “zips” (see document by Gerry et al., J. Mol. Biol., vol. 292, pp 251-262, 1999) and which have been found not to have interactions with the sequences present in the human genome in principle. This type of sequence as a Csc compound has the particular advantage of being usable without the need for modification, whatever the desired target compound.

In the course of experiments carried out in PBS 1×buffer (25 mM phosphate buffer with 0.137 MNaCl, pH 7.4), the sample of calibration target compound Ccc is firstly contacted with the biochip before the sample of target compound Cc or, inversely, the duplex formed between Csc and Ccc (or conversely between Cs and Cc) being denatured between the two series of measurements. In another embodiment, it is possible not to carry out denaturation of the first duplex formed between Csc and Ccc (or conversely between Cs and Cc). For this latter embodiment, it is preferable to place the calibration probe compound Csc between the surface and the probe compound.
The measurement system used

Reading is carried out in SPR imaging mode (SPR). The magnitude measured is the level of reflectivity (R) in TM polarisation (transverse magnetic) with respect to TE polarisation (transverse electric). This physical phenomenon gives us 6 dimensions associated with our measurements: two spatial dimensions (called x and y), one temporal (t), one spectral (λ), one angular (θ) and one polarisation dimension (TMi). The state of resonance is affected by any variation in the optical index n close to the surface, notably that caused by the hybridization of biochemical molecules and forms a film of average thickness e. Sub-nanometric resolution is sufficient for a number of applications, in particular those requiring determination of DNA sequence hybridization.

Such a measurement can be carried out in real time and makes it possible to follow the changes in a given surface over time.

In the general case of dynamic biochips where several measurements can be made sequentially and temporally, it is possible to combine a calibration step with a step to monitor the biomolecular interactions of interest. This makes it possible to correct raw experimental measurements and take into account local fluctuations in the conditions under which these measurements are made.

This involves attaching a calibration entity with the same potential specificity as during real measurement. This makes it possible to quantify the real functionality of all pixels (x, y, *) by measuring their Δn/Δe. It is then possible to take into consideration the effective lack of homogeneity of biochip surfaces, in particular the density of accessible probes, in order to interpret and quantitatively correct the dispersion observed in their behaviour during the measurement phases. As a result, this correction overcomes measurements of all causes of spatial variations that are stable in time. At the current level of relative mastery of surfaces, this makes it possible to achieve in excess of one order of magnitude in the accuracy of measurements. In general, this minimizes the limitations of the specifications schedule of the compounds in use.

### Example 2

### Results

Example of record of measurements obtained with 100 pixels of a plot (or 100 supposedly identical plots)

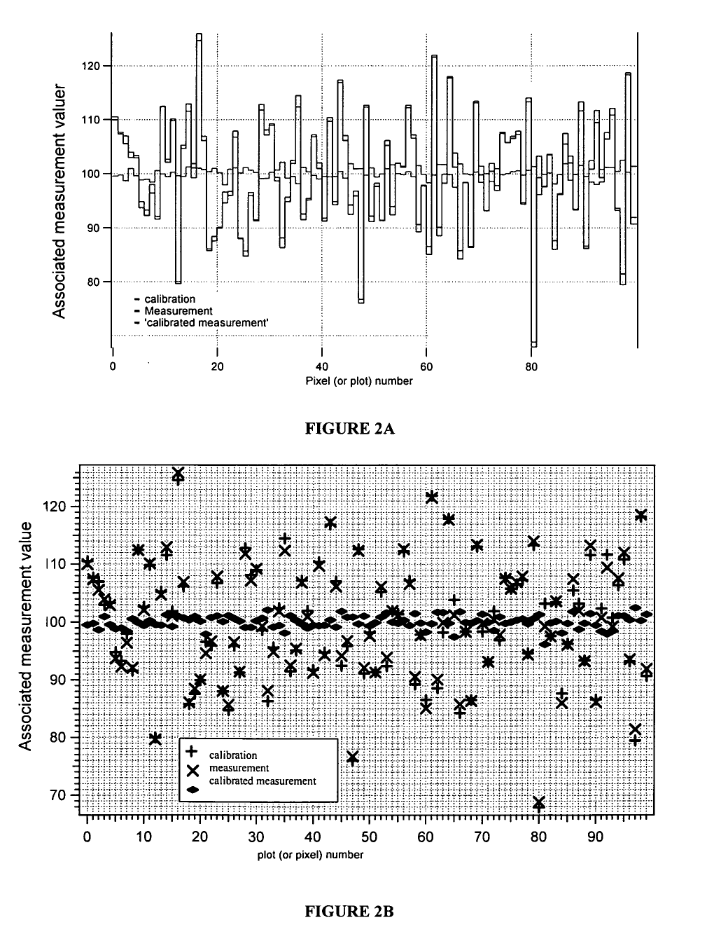

See FIGS. 2A and 2B

Record of measurements (symbol × (multiplied)) over 100 pixels of a plot (or 100 supposedly identical plots).

For the same pixels (plots), the corresponding calibration measurements are given by the symbol + (plus).

The data for measurements corrected by the variations measured in the calibration phase are represented by the symbol ♦ (diamond).

In the present case, the values for the calibration measurements are the result of Gaussian distribution centred on 100 and a magnitude of 10, those of the measurements are correlated to the calibration measurements with a Gaussian distribution of a magnitude of 1. This is in the order of magnitude that we find naturally, alone, in corrected data.

B) Histogram of raw measurements corrected by calibration measurements

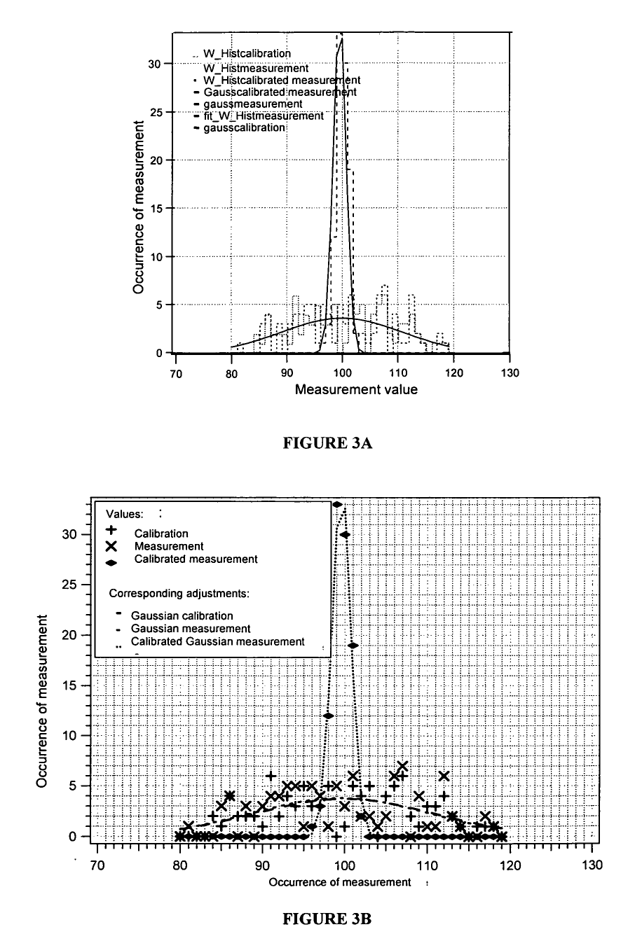

See FIGS. 3A and 3B

The histograms corresponding to such a series of measurements for 100 pixels on a plot (or 100 supposedly identical plots) are illustrated in FIGS. 3A and 3B.

The raw measurements corrected by calibration measurements are significantly less dispersed.

1. Process for determining the presence and/or quantity of a target compound Cc in a test sample, said process using a solid support to whose upper surface is attached a probe compound Cs capable of binding specifically to said compound Cc likely to be contained in the test sample, said process comprising:
a step a) consisting of contacting the test sample likely to contain said compound Cc with said support under conditions allowing the formation of specific complex Cc/Cs; and
a step b) for a dynamic label-free measurement of the formation of a specific complex Cc/Cs, possibly formed in step a),
characterised in that:
said solid support also includes attached to this upper surface a calibration probe compound Csc capable of binding specifically to a calibration target compound Ccc, the molar ratio of said Csc compound to said compound Cs being known, and in that the process is comprised of the following steps prior to or after steps a) and b):
c) contacting a sample of said compound Ccc with said support under conditions allowing the formation of a specific complex Csc/Ccc; and
d) measurement of the formation of a complex Csc/Ccc in step c) by the dynamic label-free measurement method used in step b)
the determination of the presence and/or quantity of said target compound Cc in the test sample being based on the measurements obtained in step b) and step d).

2. Process according to claim 1, characterised in that:
when steps c) and d) are carried out prior to steps a) and b), the solid support is washed under appropriate conditions after step d) in order to eliminate compound Ccc of complex Csc/Ccc from the solid support; or
when steps c) and d) are carried out after steps a) and b), the solid support is washed under appropriate conditions after step b) in order to eliminate compound Cc from complex Cs/Cc from the solid support.

3. Process according to claim 1 or 2, characterised in that steps c) and d) are carried out prior to step a).

4. Process according to one of claims 1 to 3, characterised in that a spacer compound is attached to said support, and in that compound Cs and compound Cc are attached to the support by means of said spacer compound.

5. Process according to one of claims 1 to 4, characterised in that compound Csc is attached to compound Cs by means of a covalent bond.

6. Process according to claim 5, characterised in that compound Csc is attached to the free non-attached extremity of the support of compound Csc.

7. Process according to one of claims 1 to 4, characterised in that compound Csc is attached to the surface of the solid support independently of compound Cs.

8. Process according to claim 7, characterised in that compound Csc and compound Cs are attached to the surface of the solid support by fixation to a same molecule, the latter being attached to the support.

9. Process according to claim 8, characterised in that the same molecule to which are attached compound Csc and compound Cs is a polymer.

10. Process according to one of claims 1 to 9, characterised in that:
the same compounds Cs and Csc are attached to a defined surface (called a plot) of the support;
in that the sample of compound Ccc and test sample likely to contain compound Cc of step c) are contacted with the whole of said plot of the support;
measurements in steps b) and d) are carried out for the set of points or pixels on said plot; and
determination of the presence and/or quantity of said target compound Cc in the test sample is carried out taking into account the set of measurements obtained in steps b) and d) for each of the measured points or pixels of said plot.

11. Process according to claim 10, characterised in that for a same plot, the conditions allowing the specific formation of complex Cc/Cs and Ccc/Csc are identical.

12. Process according to one of claims 10 and 11, characterised in that said support comprises n plots, n being between 2 and 10⁸.

13. Process according to one of claims 10 to 12, characterised in that compounds Csc and Ccc used are identical for n plots.

14. Process according to one of claims 10 to 12, characterised in that compounds Csc and Ccc used are different for at least 2 plots.

15. Process according to one of claims 10 to 14, characterised in that compound Cs used and compound Cc investigated are different between at least 2 plots.

16. Process according to claim 15, characterised in that the conditions allowing the specific formation of the complex Cc/Cs or Ccc/Csc are identical for 2 plots between which the compound Cs used and the compound Cc investigated are different.

17. Process according to one of claims 1 to 16, characterised in that said compound Cs and said compound Cc are nucleic acids, notably chosen from the nucleic acid group made up of double strand DNA, single strand DNA or even a mixed single and double strand type of nucleic acid, the product of transcription of said DNAs, such as RNAs, as well as any nucleic acid including or not non-natural nucleotides.

18. Process according to claim 17, characterised in that compounds Csc and Ccc are nucleic acids with no significant homology with compound Cs attached and compound Cc investigated or their complementary sequence.

19. Process according to claim 17 or 18, characterised in that compound Csc and, if need be, compound Ccc, is a nucleic acid with a length of 6 to 30 nucleotides.

20. Process according to one of claims 1 to 19, characterised in that the dynamic label-free reading method used us surface plasmon resonance (SPR) and in that said solid support is coated with a metal layer.

21. Process according to one of claims 1 to 20, characterised in that said support is a transparent solid support.

22. Process according to claim 21, characterised in that said support is a glass slide.

23. Use of a solid support comprising, attached to its upper surface, a first probe compound Cs capable of binding specifically to a target compound Cc likely to be contained in the sample, and a second probe compound Csc capable of binding specifically to another target compound Ccc, the molar ratio of said compound Csc to said compound Cs being known, said solid support being used to determine and/or quantify by means of a label-free dynamic reading method of the presence of a target compound Cc by measuring the formation of a specific complex Cs/Cc for calibration or self-calibration of said measurement after contacting the sample likely to contain the target compound Cc with the upper surface of the support.

24. Use of a solid support according to claim 23, characterised in that compound Csc is attached to one of the extremities of compound Cs by covalent coupling.

25. Use of a solid support according to claim 24, characterised in that said support is coated with a metal layer and wherein the dynamic label-free reading method used is SPR imaging.

26. Kit or necessary equipment for calibration of a measurement, or kit or necessary equipment for determination and/or quantification of the presence of a target compound Cc in a sample by measurement of the formation of a specific complex between said compound Cc and a probe compound Cs attached to the upper surface of a solid support, said compound Cs being capable of binding specifically to said target compound Cc, characterised in that measurement of the formation of complexes is carried out by means of a dynamic label-free reading method and in that the kit or necessary equipment comprises:
a)—a solid support to which is attached at the upper surface a probe compound Cs and a calibration probe compound Csc different from said probe compound Cs, the molar ratio of said compound Csc to said compound Cs being known, or if necessary
a solid support, a reagent Rs comprising compound Cs and reagent Rc comprising compound Csc, this two compounds Cs and Csc being used to for fixation to the upper surface of said support in a known molar ratio, and
b) a reagent Rcc comprising a calibration target compound Ccc, preferably of known concentration, if need be in a dry form for reconstitution or in solution, compound Ccc being capable of binding specifically to said compound Csc.

27. Kit or necessary equipment according to claim 26, characterised in that the dynamic label-free reading method used is SPR imaging and in that said solid support is coated with a metal layer.

28. Kit or necessary equipment according to claim 26 or 27, characterised in that compound Csc is coupled by covalent bonds to one of the extremities of compound Cs.

29. Device for measurement of the specific formation of a compound Cc on a solid support to which is attached at the upper surface a probe compound Cs capable of binding specifically to said target compound Cc characterised in that it includes:
said solid support also comprising attached to its upper surface a calibration probe compound Csc different from compound Cs, capable of binding specifically to a calibration target compound Ccc, different from compound Cc, the molar ratio of said compound Csc to said compound Cs being known, and said compound Csc being attached to the upper surface of said support by covalent coupling to one of the extremities of compound Cs;
a reagent comprising a calibration target compound Ccc capable of binding specifically to said compound Csc; and
a dynamic reading system of said support allowing measurement of the formation of specific complexes Cs/Cc and Csc/Ccc obtained at the upper surface of the support.

30. Device according to claim 29, characterised in that the dynamic label-free reading method used is SPR imaging and in that said solid support is coated with a metal layer.


SEQUENCE LISTING (4 sequences)

SEQ ID NO: 1 - DNA, 43 bp, Artificial sequence
  Probe sequence derived from exon 10 of non-mutated CFTR gene linked to mucoviscidosis
ttttttttttttttttttctaatgatgattagaccggtatgcg

SEQ ID NO: 2 - DNA, 43 bp, Artificial sequence
  Probe sequence derived from exon 10 of CFTR gene linked to mucoviscidosis
ttttttttttttttttttctaatggtgattagaccggtatgcg

SEQ ID NO: 3 - DNA, 43 bp, Artificial sequence
  Control probe sequence for the targeting of exon 10 of CFTR gene linked to mucoviscidosis
ttttttttttttttttttcacttcgtgccttgaccggtatgcg

SEQ ID NO: 4 - DNA, 12 bp, Artificial sequence
  Calibration sequence for control or target probe derivative from exon 10 of CFTR gene linked to mucoviscidosis
gaccggtatgcg